(19) United States
(12) Patent Application Publication
(10) Pub. No.: US 2023/0068335 A1
(43) Pub. Date: Mar. 2, 2023

(54) Graphene-conductive polymer-coated, paper-based nano-biosensor for cytokine detection

(71) Applicant: Rutgers, The State University of New Jersey, New Brunswick, NJ (US)

(72) Inventors: Ali Ashraf, McAllen, TX (US); Aaron D. Mazzeo, New Brunswick, NJ (US); Ramendra K. Pal, Hyderbad (IN); Francois Berthiaume, Metuchen, NJ (US)

(21) Appl. No.: 17/822,641

(22) Filed: Aug. 26, 2022

Related U.S. Application Data
(60) Provisional application No. 63/238,021, filed on Aug. 27, 2021.

(51) Int. Cl. G01N 33/543; G01N 27/12; G01N 27/327

(52) U.S. Cl. CPC G01N 33/5438; G01N 27/126; G01N 27/3278; B82Y 30/00

(57) ABSTRACT

Sensors and methods of fabricating sensors for detecting an analyte, such as a cytokine are provided. A sensor includes a porous, hydrophilic substrate, throughout which a coating comprising a mixture of graphene and a conductive polymer is disposed. The sensor further includes a sensing area, at which the coating is functionalized with at least one molecule that provides for a binding interaction with the analyte, and a contact area. The contact area includes an electrode in operative arrangement with the sensing area to provide a signal indicative of an impedance.

### RELATED APPLICATION

This application claims the benefit of U.S. Provisional Application No. 63/238,021, filed on Aug. 27, 2021. The entire teachings of the above application are incorporated herein by reference.

### BACKGROUND

Cytokines, a class of regulatory proteins, are essential physiological and pathological markers for diagnosis of diseases (e.g., cancer and Alzheimer's Disease) and response to injury. Tumor necrosis factor (TNF)-α (17.5 kDa) is a pro-inflammatory cytokine. A concentration of TNF-α ranges from about 10 pg/mL in healthy human serum to about 2000 pg/mL or upwards in patients with chronic wounds. Since the concentration of cytokines like TNF-α can be very small, a sensitive sensor is needed to detect cytokines.

Single-layer graphene and gold-based nano-biosensors can detect cytokines down at the pg/mL level; however, fabrication of such sensors is complicated and requires a cleanroom.

### SUMMARY

Sensors and methods of fabricating sensors that can be used for the detection of analytes, such as cytokines, are provided. Such sensors can be produced more economically than existing nano-biosensors and without cleanroom-based fabrication methods.

A sensor for detecting an analyte includes a porous, hydrophilic substrate and a coating comprising a mixture of graphene and a conductive polymer. The coating is disposed throughout the porous, hydrophilic substrate. The sensor further includes a sensing area and a contact area. The sensing area is an area at which the coating is functionalized with at least one molecule that provides for a binding interaction with the analyte. The contact area includes an electrode in operative arrangement with the sensing area to provide a signal indicative of an impedance.

The porous, hydrophilic substrate can include cellulose. For example, the porous, hydrophilic substrate can be cellulose paper, such as Whatman® paper. The coating comprising graphene and a conductive polymer can be a mixture comprising graphene nanoparticles (GNP) distributed in the conductive polymer (CP), including distributed substantially homogenously throughout the mixture. The conductive polymer can include at least one of Poly(3,4-ethylenedioxythiophene)-poly(styrenesulfonate) (PEDOT-PSS), polyaniline (PANI), polypyrrole (PPy), poly-1,5-diaminonaphthalene, and polythiophene.

The molecule at the functionalized sensing area can be a protein, peptide, polysaccharide, nucleic acid, or nucleotide sequence. For example, the protein, peptide, or polysaccharide can be an antibody, such as an antibody for a cytokine or a chemokine. The cytokine can be, for example, TNF-α, IL-6, IL-1α, IL-β, or TGF.

A method of fabricating a sensor includes coating a porous, hydrophilic substrate with a mixture comprising graphene and a conductive polymer, the coating including disposing the mixture throughout the porous, hydrophilic substrate, and functionalizing a sensing area of the coated porous, hydrophilic substrate with at least one molecule that provides for a binding interaction with an analyte. The method can further include disposing an electrode at a contact area of the coated porous, hydrophilic substrate to be in operative arrangement with the sensing area for providing a signal indicative of an impedance.

The method can include mixing graphene nanoparticles (GNP) with the conductive polymer (CP) to form the mixture, including distributing the graphene nanoparticles substantially homogenously throughout the mixture. The mixing can be performed by speed mixing with planetary motion, ultrasonication, magnetic stirring, or any combination thereof. The mixing can be of a method that applies strain to the GNP.

Functionalizing can include oxidizing the graphene disposed at the sensing area, such as by mild plasma oxidation, mild UV-ozone oxidation, or mild electrochemical oxidation. The functionalizing can include conjugating a protein, peptide, polysaccharide, nucleic acid, or nucleotide sequence to the graphene disposed at the sensing area of the coated porous, hydrophilic substrate. Where the functionalizing includes conjugation of a protein, peptide, or polysaccharide, the conjugated molecule can be an antibody. The method can further include blocking unconjugated locations of the functionalized graphene.

A method of detecting an analyte includes exposing a sensor, such as described above, to a sample, measuring an impedance of the exposed sensor, and comparing the measured impedance of the sensor to a reference impedance of the sensor to determine a presence of the analyte in the sample.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of an example sensor.

FIG. 2 is a diagram illustrating an example fabrication method for the sensor of FIG. 1.

FIG. 3 is a diagram illustrating an ink fabrication method.

FIG. 4 is a diagram illustrating graphene oxidation for use with the fabrication method of FIG. 2.

FIG. 5A is a schematic of an example interdigitated substrate for a sensor.

FIG. 5B illustrates an example interdigitated sensor.

FIG. 6A is scanning electron microscopy image (at 200 nm scale) of a graphene-conductive polymer (GR-CP) paper-based sensor.

FIG. 6B is another scanning electron microscopy image (at 200 nm scale) of a GR-CP-paper-based sensor.

FIG. 6C is a scanning electron microscopy image (at 100 nm scale) of a GR-CP-paper-based sensor.

FIG. 6D is another scanning electron microscopy image (at 100 nm scale) of a GR-CP-paper-based sensor.

FIG. 6E is a Raman microscopy spectrum of the sensor of FIGS. 6A-D before plasma oxidation.

FIG. 6F is a Raman microscopy spectrum of the sensor of FIGS. 6A-D after plasma oxidation.

FIG. 7A illustrates electrochemical impedance spectroscopy (EIS)-based results of an example sensor after different steps of fabrication and ink-coating of the substrate, plasma oxidation, and EDC-NHS chemistry. The plasma oxidized sample (circle) shows higher impedance than just ink coated sample (square). Similarly, EDC-NHS chemistry (upward triangle) also increases the impedance of the plasma oxidized sample. An unoxidized sample after EDC-NHS chemistry (downward triangle) does not significantly differ from just ink coating, indicating poor NHS functionalization.

FIG. 7B is a graph of cyclic voltammetry results of the sensor before and after plasma oxidation. The plasma oxidized sample was less conductive, indicating oxygen functional group formation.

FIG. 8A illustrates EIS-based results of TNF-α antigen detection using an example GR-CP-paper-based sensor. The sensor was able to distinguish between different concentrations due to change in semicircular shape (of the EIS curve) resulting from change in charge transfer resistance.

FIG. 8B is a graph of normalized charge transfer resistance of the sensor versus different TNF-α concentrations.

FIG. 8C illustrates EIS-based results of TNF-α antigen detection in PBS using an example GR-CP-paper-based interdigitated sensor (inset photo) with baseline (square), 5 minute (circle), and 20 minute (upward triangle) results plotted.

### DETAILED DESCRIPTION

A description of example embodiments follows.

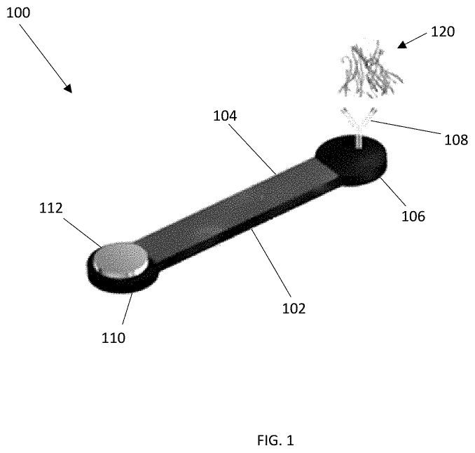

A sensor for detecting an analyte is shown in FIG. 1. The sensor 100 includes a porous, hydrophilic substrate 102 that is coated with a mixture of graphene and a conductive polymer. The coating is disposed throughout or substantially throughout the porous, hydrophilic substrate. A passivation layer 104 can be included on at least a portion of the sensor. A sensing area 106 is included at which the coating is functionalized with at least one molecule 108 capable of providing for a binding interaction with an analyte 120. The sensor 100 further includes a contact area 110 that includes an electrode 112 in operative arrangement with the sensing area 106 to provide a signal indicative of an impedance of the device. The passivation layer 104 can prevent liquid containing the analyte from travelling to the contact area 110 from the sensing area 106 due to wicking action of the porous substrate (e.g., paper). If a liquid sample interacts with a contact pad (e.g., contact area 110), an impedance measurement can contain error. As illustrated in FIG. 1, the sensor 100 is of a generally elongated shape, with the sensing area 106 and contact area 110 disposed at opposing ends of the device. Other operative arrangements are possible. For example, the sensing area 106 can be of a larger proportional area and can, optionally, be of a digitated shape, as shown in FIGS. 5A and 5B.

In particular, a sensor 500 can comprise a porous, hydrophilic substrate 502 (e.g., a paper layer) that is cut or otherwise formed to provide for one or more interdigitated sensing area(s) 506, as shown in FIG. 5A. The sensor 500 is shown in FIG. 5B upon coating of the substrate 502 with a mixture of graphene and a conductive polymer, and application of passivation layer(s) 504 and electrodes 512. In the illustrated example, electrodes 512 are arranged to provide a counter electrode (CE) and a working electrode (WE). The interdigitated sensing area(s) 506 can be functionalized, as described further below.

Interdigitated sensing areas can potentially provide for higher sensitivity as compared to bulk sensing area configurations. Examples of interdigitated configurations are further described in Van Gerwen et al. (Van Gerwen, P., Laureyn, W., Laureys, W., Huyberechts, G., De Beeck, M. O., Baert, K., Suls, J., Sansen, W., Jacobs, P., Hermans, L. and Mertens, R., 1998. Nanoscaled interdigitated electrode arrays for biochemical sensors. Sensors and Actuators B: Chemical, 49(1-2), pp. 73-80), the entire teachings of which are incorporated herein by reference.

As used herein, the term “binding interaction” is an attractive interaction between two molecules that results in a stable association, including, for example, a chemical interaction (e.g., an interaction between an antibody and antigen, an interaction between a ligand and a receptor, etc.).

The porous, hydrophilic substrate can comprise cellulose. For example, the substrate can be cellulose paper, such as Whatman® filter paper.

The mixture of graphene and a conductive polymer can include graphene nanoparticles that are distributed substantially homogenously in the conductive polymer. Examples of suitable conductive polymers include poly (3,4-ethylenedioxythiophene)-poly(styrenesulfonate) (PEDOT-PSS), polyaniline (PANI), polypyrrole (PPy), and poly-1,5-diaminonaphthalene, polythiophene.

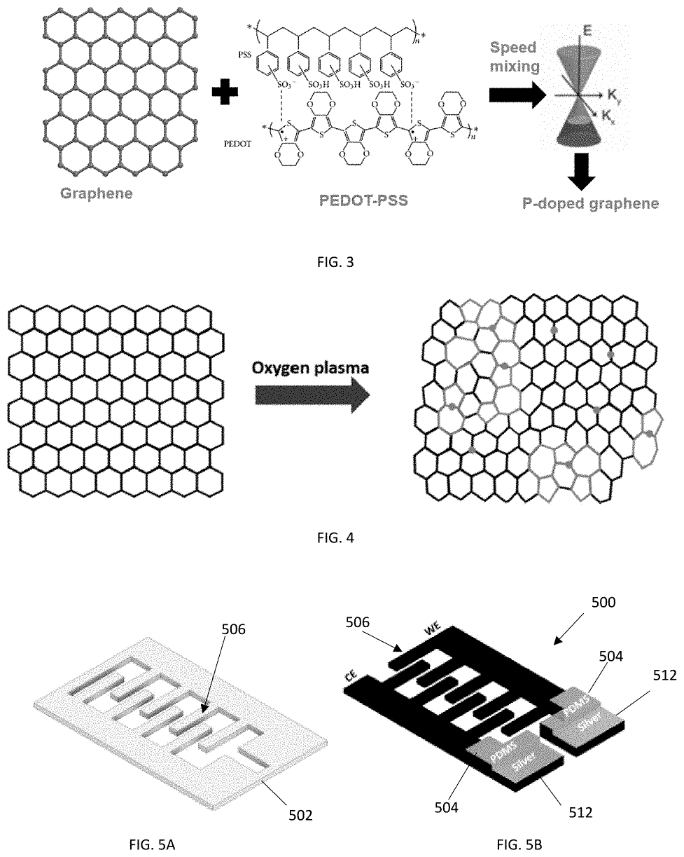

A method of making the mixture (alternatively referred to as “ink”) for use with fabrication of a sensor is shown in FIG. 3. As illustrated, graphene particles, such as graphene nanoflakes (GNF), and conductive polymer (CP) material, such as PEDOT-PSS, can be mixed by a method capable of providing a substantially homogenous or uniform mixture. The mixing method can be one that induces strain on the graphene particles. Suitable mixing methods include speed mixing with planetary motion, ultrasonication, and magnetic stirring. Such mixing methods can ensure uniform dispersion and interaction of the GNF with the CP material. The resulting ink comprises p-doped graphene. The resulting ink can be of a suitable viscosity for application to the substrate by methods such as dip coating and screen printing. The ink can be capable of permeating throughout the substrate such that the substrate is saturated by the ink.

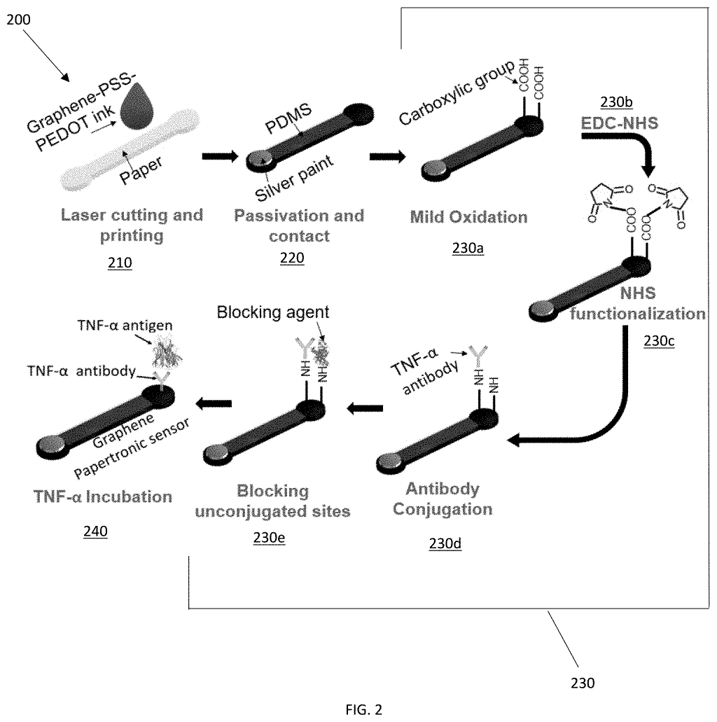

A method of fabricating a sensor is shown in FIG. 2. The method 200 includes coating a porous, hydrophilic substrate (e.g., cellulose paper) with a mixture, such as an ink produced with the method shown in FIG. 3 as illustrated at step 210. The mixture comprises graphene and a conductive polymer (e.g., Graphene-PSS-PEDOT ink). The paper can be, for example, laser cut and printed with the “ink” mixture, in either order. The method further includes disposing an electrode at a contact area of the coated porous, hydrophilic substrate, as illustrated at step 220, such as through application of a layer of silver (e.g., silver paint) at the contact area, and passivating the device, such as through application of a layer of Polydimethylsiloxane (PDMS) to exposed areas of the device other than the contact and sensing areas. The method further includes functionalizing the sensor, as shown generally at step 230.

As used herein, the term “functionalizing’ means providing for modification of a surface, so as to adapt the surface for an intended use. The functionalization of a sensor, as shown in FIG. 2 includes adhering or conjugating at least one molecule that provides for a binding interaction with an analyte to the sensing area of the sensor. The molecule can be, for example, a protein, peptide, polysaccharide, nucleic acid, or nucleic acid sequence. The protein can be an antibody, such as a TNF-α antibody or an antibody for other cytokines (e.g., IL-6, IL-1α, IL-β, TGF), chemokines, enzymes, and bioactive peptides. In one example, the at least one molecule is a TNF-α antibody and the analyte is a TNF-α antigen.

The functionalization of the sensor can optionally include oxidizing the sensing area (step 230a). A method of oxidizing the sensing area includes exposing the sensing area to plasma oxidation or electrochemical oxidation, which can introduce defects or distortions to graphene layers of the sensor, as schematically illustrated in FIG. 4, and introduce carboxylic groups to the sensing area surface for use by subsequent coupling chemistry. In one example, the plasma oxidation can be a mild plasma oxidation involving limited power, pressure, and/or exposure to the electrolyte (e.g., 18 watt power, 150 millitorr pressure, 10 seconds exposure). Examples of electrochemical oxidation are described in Rengaraj, Saravanan, et al. “Impedimetric paper-based biosensor for the detection of bacterial contamination in water,” Sensors and Actuators B: Chemical 265 (2018): 50-58, the entire teachings of which are incorporated herein by reference. Examples of plasma oxidation treatments are described in Ryu, Gyeong Hee, et al. “Effects of dry oxidation treatments on monolayer graphene,” 2D Materials 4.2 (2017), the entire teachings of which are incorporated herein by reference.

The functionalization of the sensor can include further coupling chemistry for the molecule to be conjugated to the sensor (steps 230b-d). As illustrated in FIG. 2 with respect to the example coupling of a TNF-α antibody, coupling chemistry involving EDC (1-Ethyl-3-(3-dimethylaminopropyl)carbodiimide)-NHS (N-Hydroxysuccinimide), which makes use of carboxylic functional groups on the graphene surface, can be used. Optionally, upon conjugation of the molecule (e.g., TNF-α antibody), unconjugated sites can be blocked, e.g., using a blocking agent, as illustrated at step 230e of FIG. 2. Coupling of an antibody can also be provided by other methods, such as, for example EDC-only methods and EDC-sulfo NHS methods. See, for example, Vashist, S. K., 2012 (“Comparison of 1-ethyl-3-(3-dimethylaminopropyl) carbodiimide based strategies to crosslink antibodies on amine-functionalized platforms for immunodiagnostic applications.” Diagnostics, 2(3), pp. 23-33), the entire teachings of which are incorporated herein by reference.

The resulting paper-based sensor (step 240) can then be used for the detection of the analyte of interest (e.g., TNF-α antigen) using electrochemical impedance spectroscopy (EIS). For example, an inherent impedance of a fabricated sensor can be measured when the sensor is exposed to a baseline solution (e.g., phosphate buffer saline (PBS) solution or PBS with ferri-ferro cyanide). This inherent impedance can serve as a reference impedance, which can be compared to an impedance as measured upon contact of the sensor to a test sample for detection of an analyte of interest.

The described sensors and methods of fabricating such sensors can be used for the detection of cytokines, which are a class of regulatory proteins that can be important physiological and pathological markers for prognosis and diagnosis of various diseases, such as cancer and Alzheimer's Disease, as well as for monitoring response to an injury, which can induce a cytokine response.

As a concentration of cytokines, such as TNF-α, can be very small, sensor sensitivity can be needed for cytokine detection. Other sensors, such as single-layer graphene or gold-based nano-biosensors, can detect cytokines down at the pg/mL level; however, their fabrication is complicated and requires cleanroom. Furthermore, a PEDOT-PSS firm or GNF film formed on cellulose fibers is not itself stable. In particular, PSS undergoes swelling, and a GNF film undergoes weight loss when exposed to liquid. However, their combination can form a stable film for long-term sensing performance due to stronger bonding between PEDOT-PSS and graphene. Additionally, GNF can be easily altered using mild plasma to conjugate antibodies. The sensors, and methods of making such sensors provided herein provide for significant improvements over existing sensors, including significantly easier fabrication and improved sensitivity.

The use of a planetary motion mixer in the preparation of the conductive ink from a graphene nanoflake (GNF)-conductive polymer (CP) mixture provides for several advantages over conventional methods of fabricating paper-based biosensors. This mixing technique can ensure uniform dispersion and interaction of GNF with CP. For detection, label-free electrochemical impedance spectroscopy (EIS) can be used, making this technology suitable for point-of-care applications. Such fabrication methods, which can make use of techniques such as screen printing, can provide for economically viable and flexible paper-based biosensors for sensitive detection of cytokines.

Cellulose is an abundantly available biopolymer that is renewable, biocompatible, and biodegradable, while also having high stiffness, strength, thermal stability, and sorption capacity. Paper-based electrochemical sensors, such as the sensors provided herein, can enable point-of-care (POC) diagnostics for various disease applications, and can be particularly suitable for detecting and/or characterizing wound healing. Paper-based electrochemical sensors do not require a cleanroom, nor complicated, expensive conventional micro/nano-fabrication techniques. Furthermore, electrochemical impedance spectroscopy (EIS) with use of paper-based sensors, as provided herein, can provide high selectivity and sensitivity.

### EXEMPLIFICATION

### Example 1

### Sensor for Detection of TNF-α Antigen

Concentration of tumor necrosis factor (TNF)-α (17.5 kDa), a pro-inflammatory cytokine, ranges from 10 pg/mL in healthy human serum, to ˜300 pg/mL in patients with autoimmune disease.

Prototype sensors were fabricated using dip coating methods with a conductive ink and commercial paper substrates (Whatman® filter paper), which demonstrated measurement of TNF-α from 0.8-800 pg/mL using electrochemical impedance spectroscopy based immunosensing in the frequency range of 10 kHz to 100 mHz. Additional details regarding the fabrication of prototype sensors and results obtained during testing with such sensors can be found in Examples 2 and 3 herein.

This work presented a facile method of fabricating paper-based nano biosensors using conductive ink composed of graphene nanoflakes (GNF) and Poly(3,4-ethylenedioxythiophene)-poly(styrenesulfonate) (PEDOT-PSS) conductive polymer for the detection of cytokines. The viscosity and stability of the conductive ink was improved through adjusting the ratio of GNF and PEDOT-PSS in DMSO (Dimethyl Sulfoxide) solvent. Additionally, proper mixing of the ink components was achieved by using a FlackTek® speedmixer. During this mixing process, GNF can curl up, and additionally get doped with PEDOT-PSS through π-π interactions, thereby increasing the charge carrier mobility of GNF and hence sensitivity for biosensing. The results show that Whatman® filter paper with larger porosity than Fabriano® paper provided a stable conductive film by providing pathways to connect top and bottom conductive layers through the open pores. Paper-based substrates were coated multiple times to improve the stability of the coated film and control the channels for ion conduction in the nano-biosensor. Using brief atmospheric plasma-based dry oxidation, the number of active sites on the sensor surface for TNF-α antibody attachment was increased via EDC-NHS chemistry. To determine the presence and distribution of GNF and PEDOT-PSS, sensor substrates were characterized by scanning electron microscopy and Raman spectroscopy. This research paves the way towards economically viable and flexible paper-based biosensors using techniques like screen printing for sensitive detection of cytokines.

Paper-based nano-biosensors have many advantages in terms of cost, biodegradability, flexibility compared to conventional sensors based on silicon technology. The preparation of uniform conductive ink from graphene nanoflake (GNF)-conductive polymer (CP) mixture using a planetary motion mixer provided uniform dispersion and interaction of GNF with CP. After coating of the filter paper substrate, mild plasma oxidation was utilized to impart carboxyl functional groups on GNF surface, necessary for antibody conjugation. For detection, a label-free electrochemical impedance spectroscopy (EIS) technique was provided, making this technology suitable for point-of-care application. Sensors prepared in this fashion showed high sensitivity (picogram/milliliter level) while detecting cytokine, thereby making this technology suitable for disease detection and wound monitoring.

### Example 2

### Experimental Design

In this example, 1 g of GNF (Cheap Tubes, VT) was mixed with 0.1 g of poly(3,4-ethylene dioxythiophene) polystyrene sulfonate (PEDOT-PSS) conductive polymer (Sigma Aldrich, Mo.) dissolved in 6 mL of deionized water and 800 μL of dimethyl sulfoxide (DMSO) solvent in a FlackTek® Speedmixer to prepare the biosensor ink. Subsequently, this well-mixed ink was deposited on a laser patterned Whatman® filter paper through drop-casting.

Silver paint was used to construct the electrical contact pad and PDMS coating to separate the testing area from the contact area. Cellulose is well known to absorb water and provide a diffusion pathway for ions to flow from the testing area to the contact pad. Therefore, it can be crucial to use hydrophobic PDMS coating to avoid noisy and erroneous data collection.

The next step was to attach the TNF-α antibody to the graphene surface. However, the coupling chemistry using EDC (1-Ethyl-3-(3-dimethylaminopropyl)carbodiimide)-NHS (N-Hydroxysuccinimide) makes use of carboxylic functional groups on the graphene surface. To preserve structural integrity and introduce needed oxygen functional groups on the graphene surface, mild dry plasma oxidation was performed on the sensors at 150 mtorr for 10 seconds at 20 watts.

After that, the sensor surface was activated using EDC-NHS coupling chemistry and conjugated the TNF-α antibody to the sensor surface by overnight incubation at 4° C.

Before TNF-α antigen detection, the unreacted sites were blocked using Tween 20 surfactant.

Antigen detection testing was performed using electrochemical impedance spectroscopy (EIS). After exposing the sensor to different antigen concentrations in phosphate-buffered saline (PBS) solution for ˜40 minutes, electrical impedance was measured in the frequency range of 10000-0.1 Hz using a PARSTAT 3000 potentiostat. During measurement, a 2 mM Ferri-Ferro-phosphate buffer saline (PBS) solution was used. It has been shown that a 2 mM Ferri-Ferro solution can reduce errors that might result from variation in charge transfer resistance.

Raw data from the tested sensors were fitted to a modified Randles circuit: electrolyte bulk resistance Rs, electrolyte in the pore resistance R1, charge transfer resistance Rct, electrolyte (in pore) capacitance C1, constant phase element (sensor surface) Q and Warburg diffusion element W. The ZSimpWin 3.60 (AMETEK, USA) software was used to build and verify this model.

The morphology of cold-fractured porous electrodes was investigated using a Zeiss field emission scanning electron microscopy (SEM). Raman data were collected using a 633 nm laser using a Renishaw inVia reflex system with 50× magnification.

### Example 3

### Results

Graphene nanoflakes (GNF) along with conductive polymer (CP) were utilized to coat the filter paper uniformly. Microwave-assisted or liquid exfoliated GNF is simpler to prepare than graphene produced by chemical vapor deposition. The steps used to fabricate a GNF-CP-Paper-based biosensor are shown in FIG. 2.

As a first step, GNF were mixed with CP in a mixer having planetary motion to prepare the conductive ink. The porous and hydrophilic nature of the Whatman® filter paper ensures uniform coating of the cellulose fibers by GNF-CP ink. The choice of ink components, mixing technique, and substrate are deemed critical for a stable and effective sensor. Film delamination was observed on hard ITO and hydrophobic Fabriano® paper substrates. Additionally, it was found that if the ink dries too quickly due to the application of high temperature (˜70 C), the film will delaminate. The filter paper's micro-nano porous morphology and hydrophilic nature ensured uniform coating and interaction between the ink and cellulose fibers.

Cyrene was tried as a solvent for GNF-CP instead of DMSO. However, the film's conductivity with Cyrene was low compared to the film prepared with DMSO. The addition of DMSO ensured better cohesion and electrical properties due to the inter-PEDOT bridging mechanism.

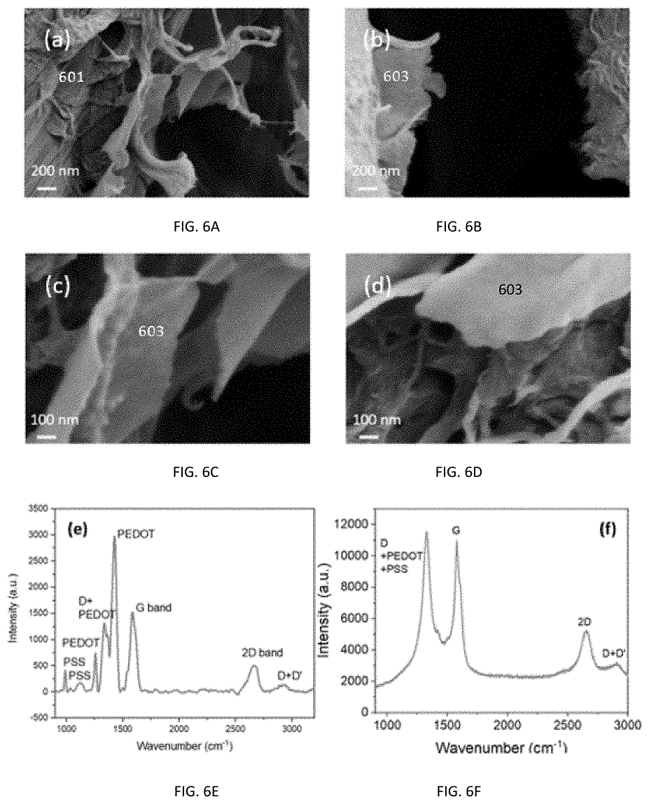

A Flacktek® mixer was used to achieve uniform mixing and introduce curvature to the GNF. The Dual Asymmetric Centrifuge of the mixer ensured uniform mixing, removal of bubbles, introduced curvature-induced strain to the GNF and π-π* interaction of GNF with PEDOT-PSS, leading to enhancement of charge carrier concentration of GNF (doping). The enhanced affinity of CP to cellulose fibers and interaction of CP with GNF allowed GNF to be glued to the fibers. PEDOT-PSS self-assembles on the fibril in the wet stage gets π-stacked after drying and, therefore, acts as a glue to connect graphene with cellulose fibers. This can be observed from the SEM images of GNF-CP-paper-based sensor, as shown in FIGS. 6A-6D. The flat GNF sheets (603) are connected to the fibrous structure of cellulose (601). The hierarchical structure with more prominent pores and fibers was observed in low magnification SEM images. Experiments were conducted with more viscous ink (containing double the amount of PEDOT-PSS compared to original ink), which led to the formation of a thick crust on the surface, preventing access to the micro-nano porous structure underneath. Therefore, ink with less PEDOT-PSS was used. However, to ensure uniform coating of the entire substrate, the sensor was multicoated (4 times) and dried, which did not lead to crust formation.

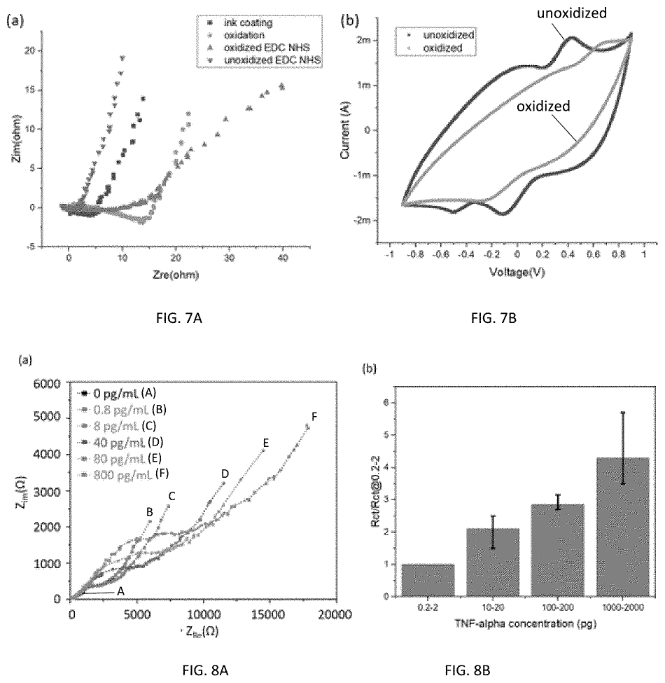

To perform EDC-NHS chemistry on the sensor surface, carboxylic function groups (—COOH) were needed. GNF may have some oxygen functional groups on the edge sites of nanoflakes formed during manufacturing, but their amount was likely to be minimal to perform the reaction. Therefore, mild oxygen plasma treatment (˜20 W for 10 s) was used to functionalize the GNFs by creating defect sites that form oxygen functional groups when exposed to ambient conditions. This was done following the methodology presented by Ryu et al. (G. H. Ryu, J. Lee, D. Kang, H. J. Jo, H. S. Shin, and Z. Lee, “Effects of dry oxidation treatments on monolayer graphene,” 2D Mater., vol. 4, no. 2, p. 024011, June 2017). In particular, dry oxidation was used, which is an effective means for introducing topological defect and lattice distortion to the monolayer graphene surface, which leads to the attachment of oxygen atoms to the defect site. Successful oxidation can be verified by Raman spectroscopy, which can show signature peaks from CP and GR, and, additionally, an amount of defect and functionalization (D band). This mild oxidation treatment led to broadening of the D peak (˜1350 cm−1), as can be observed from before and after plasma treatment Raman spectra (FIGS. 6E-6F). Additionally, electrochemical impedance spectroscopy (EIS) and cyclic voltammetry (CV) of the sensor after different processing steps were performed, shown in FIGS. 7A and 7B, respectively. The plasma oxidized sample shows higher resistance or lower conductivity than the unoxidized sample, indicating suitable oxygen functional group formation for antibody conjugation by EDC-NHS chemistry.

In studying this graphene-conductive polymer-paper-based biosensor, Tumor Necrosis Factor α (TNF-α) was selected as a model cytokine. Therefore, the TNF-α antibody was used to conjugate with NHS functional group.

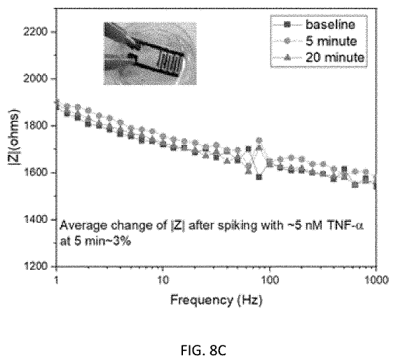

In this work, TNF-α level in PBS were measured. The EIS-based testing of the sensor with different concentrations of TNF-α antigen is shown in FIG. 8A. The sensor distinguished between different concentrations due to a change in semicircular shape resulting from a change in charge transfer resistance. FIG. 8B shows the normalized charge transfer resistance (RCT) vs. TNF-α concentration from testing three different sensors. The sensor does show small batch to batch variation, which can happen due to manual preparation and coating of the ink and change in ambient conditions. FIG. 8C illustrates results with an interdigitated sensor.

The sensor was prepared with ink that only contained CP. However, the sensor with only CP was not stable during multiple EIS measurements, whereas the sensor with GR-CP showed stable results during multiple runs.

The raw EIS data were modeled using ZSimpWin software to determine RCT. The data points became noisy at low frequency and the fit was slightly biased by the data points collected during high frequency.

Additionally, the effect of sensor geometry and the importance of Ferro-Ferri probe during sensing experiment were investigated. It was found that if the sensor area is decreased (thin finger sensor) sensitivity decreases, and the same phenomenon was observed when no Ferro-Ferri probe was present.

Finally, the potential of non-specific interaction of the sensor was investigated by testing the sensor for cortisol detection at low (5 pM) and high (5 nM) concentrations. The sensor showed minimal change unlike when it was exposed to similar amount of TNF-α, indicating the sensor has good selectivity.

It was found that the Graphene nanoflake-PEDOT-PSS-paper based sensor was able to detect TNF-α from 0.2-2000 pg/mL range. Hydrophilic porous Whatman® filter paper was found to provide for a stable conductive layer, as opposed to other types of substrates tested.

The teachings of all patents, published applications and references cited herein and in the attached appendix are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A sensor for detecting an analyte, comprising:
a porous, hydrophilic substrate;
a coating comprising a mixture of graphene and a conductive polymer, the coating disposed throughout the porous, hydrophilic substrate;
a sensing area at which the coating is functionalized with at least one molecule that provides for a binding interaction with the analyte; and
a contact area comprising an electrode in operative arrangement with the sensing area to provide a signal indicative of an impedance.

2. The sensor of claim 1, wherein the porous, hydrophilic substrate comprises cellulose.

3. The sensor of claim 1, wherein the porous, hydrophilic substrate is a cellulose paper.

4. The sensor of claim 1, wherein the coating comprising graphene and a conductive polymer is a mixture comprising graphene nanoparticles distributed in the conductive polymer.

5. The sensor of claim 4, wherein the graphene nanoparticles are substantially homogenously distributed in the mixture.

6. The sensor of claim 1, wherein the conductive polymer comprises at least one of Poly(3,4-ethylenedioxythiophene)-poly(styrenesulfonate) (PEDOT-PSS), polyaniline (PANI), polypyrrole (PPy), poly-1,5-diaminonaphthalene, and polythiophene.

7. The sensor of claim 1, wherein the molecule is a protein, peptide, polysaccharide, nucleic acid, or nucleotide sequence.

8. The sensor of claim 7, wherein the protein, peptide, or polysaccharide is an antibody.

9. The sensor of claim 8, wherein the antibody is an antibody for a cytokine or a chemokine.

10. The sensor of claim 9, wherein the antibody is an antibody for a cytokine and the cytokine is selected from the group consisting of TNF-α, IL-6, IL-1α, IL-1β, and TGF.

11. A method of fabricating a sensor, comprising:
coating a porous, hydrophilic substrate with a mixture comprising graphene and a conductive polymer, the coating including disposing the mixture throughout the porous, hydrophilic substrate;
functionalizing a sensing area of the coated porous, hydrophilic substrate with at least one molecule that provides for a binding interaction with an analyte; and
disposing an electrode at a contact area of the coated porous, hydrophilic substrate to be in operative arrangement with the sensing area for providing a signal indicative of an impedance.

12. The method of claim 11, further comprising mixing graphene nanoparticles with the conductive polymer to form the mixture.

13. The method of claim 12, wherein the mixing comprises distributing the graphene nanoparticles substantially homogenously throughout the mixture.

14. The method of claim 12, wherein the mixing is performed by at least one of the following: speed mixing with planetary motion, ultrasonication, and magnetic stirring.

15. The method of claim 12, wherein the mixing includes applying strain to the graphene nanoparticles.

16. The method of claim 11, wherein functionalizing the sensing area includes oxidizing the graphene disposed at the sensing area of the coated porous, hydrophilic substrate.

17. The method of claim 16, wherein the oxidizing is performed by mild plasma oxidation or mild electrochemical oxidation.

18. The method of claim 11, wherein functionalizing the sensing area includes conjugating a protein, peptide, polysaccharide, nucleic acid, or nucleotide sequence to the graphene disposed at the sensing area of the coated porous, hydrophilic substrate.

19. The method of claim 18, wherein the conjugating is of a protein, peptide, or polysaccharide and the protein, peptide, or polysaccharide is an antibody.

20. The method of claim 18 further comprising blocking unconjugated locations of the functionalized graphene.

21. A method of detecting an analyte, comprising:
exposing the sensor of claim 1 to a sample;
measuring an impedance of the exposed sensor; and
comparing the measured impedance of the sensor to a reference impedance of the sensor to determine a presence of the analyte in the sample.
